(19) United States
(12) Patent Application Publication
(10) Pub. No.: US 2011/0044963 A1
(43) Pub. Date: Feb. 24, 2011

(54) Method of Testing a Patient for Hypertension and Related Method of Treatment and Test Kit

(76) Inventor: Jules B. Puschett, Temple, TX (US)

Correspondence Address: ECKERT SEAMANS CHERIN & MELLOTT, 600 GRANT STREET, 44TH FLOOR, PITTSBURGH, PA, 15219, US

(21) Appl. No.: 12/706,021

(22) Filed: Feb. 16, 2010

Related U.S. Application Data
(60) Provisional application No. 61/158,474, filed on Mar. 9, 2009.

(51) Int. Cl. A61K 38/43; G01N 33/00; G01N 33/52

(52) U.S. Cl. 424/94.1; 436/86; 422/430

(57) ABSTRACT

A method of testing a patient for hypertension includes providing a urine specimen from the patient, performing a first test on the urine specimen to determine if volume expansion hypertension exists by measuring the level of marinobufagenin in the urine specimen, performing a second test on the urine specimen to determine if vasoconstrictive hypertension exists by measuring the level of angiotensinogen in the urine specimen, and performing the first and second tests on the urine specimen substantially simultaneously without either test having an adverse effect on the other test. The tests may employ a test strip having a first region for use in the first test and a second region for use in the second test with each region effecting a visible change if the condition for which the test is being formed is found to exist. A related method of treatment and a related test kit are also disclosed.

### CROSS-REFERENCE TO RELATED APPLICATION

The present application is a traditional application of U.S. Provisional Patent Application Ser. No. 61/158,474, filed Mar. 9, 20108, and entitled, “Method of Testing a Patient for Hypertension and Related Method of Treatment and Test Kit,” which is herein incorporated by reference in its entirety.

### BACKGROUND OF THE INVENTION

1. Field of the Invention

The present invention relates to a diagnostic method for determining the presence of volume-dependent hypertension or vasoconstrictive hypertension in a urine specimen, and more specifically, it provides for substantially simultaneous performance of tests for each of these categories of hypertension, along with a method of treatment and a test kit.

2. Description of the Prior Art

Elevated blood pressure, or hypertension, has long been recognized as a serious health problem. It is a very common disease, which can have wide-spread, detrimental effects on a patient, and frequently, unlike numerous other medical conditions, is asymptomatic.

From a pathogenetic standpoint, essential hypertension may be divided into two categories: (a) volume expansion hypertension and (b) vasoconstrictive hypertension. It has been estimated that about 30% to 40% of human essential hypertension may be primarily related to volume expansion hypertension especially in certain demographic groups.

Despite the known means for measuring blood pressure of a patient, as by a sphygmomanometer, for example, there is lacking an accurate, reliable means for detecting, substantially simultaneously from a urine specimen, the existence of volume-dependent hypertension and vasoconstrictive hypertension.

United States Patent Application Publication No. 2008/0026409 discloses a method and related kit for hypertension involving the identification of compositions of biomarkers, which distinguish between salt-sensitive hypertension, salt-resistant hypertension, salt-independent hypertension, and other hypertensive disorders. The disclosure states that the development identifying varying levels of biomarkers, other hypertensive disorders, and varying levels of biomarkers in a patient, as compared with normal patients. It also discloses pharmaceutical agents employed to treat patients.

U.S. Pat. Nos. 5,374,525 and 5,589,584 disclose testing for polymorphisms/mutations in the angiotensinogen gene with a view toward ascertaining predisposition to hypertension.

U.S. Pat. No. 6,632,180 is directed toward the evaluation and treatment of hypertension based upon the determination of plasma renin activity with a view toward enhancing the treatment of hypertension.

U.S. Pat. No. 4,690,907 discloses an immunoassay for detecting the presence of a target substance in a sample. It employs capillarity by having the reaction product or any remaining unreacted immunoreactory agent combined with a substance packed in a capillary tube resulting in measurement of the amount of immobilized labeled substance.

U.S. Pat. No. 6,251,611 is directed toward a method of determining volume-dependent hypertension by monitoring reduction in phosphorylation of blood-derived protein or renal proximal brush border membrane proteins by employing blood serum or blood plasma and related therapeutic patient treatment and diagnostic apparatus.

In spite of the existing prior art, there remains a need for an effective method and related kit for substantially simultaneously employing a urine specimen to ascertain whether a patient has volume expansion mediated hypertension through monitoring of bufodienolides, such as marinobufagenin in the urine, and determining if a patient has vasoconstrictive hypertension through monitoring the angiotensinogen in the urine. Prompt therapeutic measures can then be taken.

### SUMMARY OF THE INVENTION

The present invention has met above-described needs by providing a method for testing a patient for hypertension comprising providing a urine specimen from the patient, performing a first test on the urine specimen to determine if volume expansion hypertension exists by measuring the level of a bufodienolide in the urine specimen, performing a second test on the urine specimen to determine if vasoconstrictive hypertension exists by measuring the level of angiotensinogen in the urine specimen, and performing the first and second tests on the urine specimen substantially simultaneously without either test having an adverse effect on the hypertension measurement of the other test. After determining the type of hypertension which exists, treatment of the patient may be employed.

The bufodienolide may be marinobufagenin

The tests may be performed on the urine in the same container substantially simultaneously.

A test kit may provide a test strip having at least one region for testing for volume expansion hypertension and may provide a visual indication, either with the naked eye or through a proper instrument, of whether volume expansion hypertension exists. A second region of the test strip may provide an indication based upon the level of angiotensinogen in the urine as to whether vasoconstrictive hypertension exists.

In another embodiment of the present invention, the urine is tested for the level of bufodienolide, such as marinobufagenin, in order to determine if volume expansion hypertension exists.

It is an object of the present invention to provide a method of determining whether a patient has volume expansion hypertension or vasoconstrictive hypertension employing urine as a specimen.

It is a further object of the present invention to employ the level of a bufodienolide, such as marinobufagenin, in making the determination regarding whether volume expansion hypertension exists and employing the level of angiotensinogen in determining whether vasoconstrictive hypertension exists.

It is a further object of the present invention to provide a kit which may employ a test strip having separate regions for determination of the presence of volume expansion hypertension and vasoconstrictive hypertension through immersion of a strip in a urine specimen and subsequent visual inspection to determine whether a sufficient change in a region of the test strip indicates the presence of either type of hypertension.

It is a further object of the present invention to facilitate simultaneous testing for both of these types of hypertension with a rapid response permitting immediate, targeted treatment of the type of hypertension, as by administering appropriate pharmaceuticals.

It is another object of the present invention to provide a kit, wherein a single test strip may be employed to simultaneously test a patient's urine specimen for both volume expansion hypertension and vasoconstrictive hypertension without either test interfering with the accuracy and validity of the other test.

These and other objects of the present invention will be more fully understood from the following description of the invention with reference to the drawings appended hereto.

### BRIEF DESCRIPTION OF THE DRAWING

FIG. 1 is a front elevational view of a form of test strip employable in the method and kit of the present invention.

FIG. 2 is a right-side elevation of the test strip of FIG. 1.

### DETAILED DESCRIPTION OF THE PREFERRED EMBODIMENTS

As employed herein, the term “patient” refers to a human being.

As employed herein, the term “bufodienolide” means marinobufagenin and other bufodienolides.

As employed herein, the term “angiotensinogen” refers to a peptide that is converted by the enzyme renin to angiotensin I, which is a decapeptide, which is converted to angiotensin II, an octapeptide by the angiotensin-converted enzyme (“ACE”).

By testing urine for the presence and level of bufodienolides, such as marinobufagenin, one may obtain a reliable diagnostic response to whether a patient has volume expansion hypertension. Bufodienolides are a family of steroids which were first noted in the skin and venom of the common toad, Bufo marinus. They are also present in plants and circulate in blood and are excreted in the urine of humans. Among the congeners of marinobufagenin are resibufogenin, which differs from marinobufagenin only in the absence of an hydroxyl group at the beta-5 position. The preferred compounds are marinobufagenin and its congeners.

Angiotensinogen is a peptide that is converted by an enzyme, renin, to angiotensin I, which is a decapeptide, which is then converted to the active form angiotensin II, which is an octapeptide by an angiotensin-converting enzyme (“ACE”).

In employing angiotensinogen to ascertain whether vasoconstrictive hypertension exists, the measurement may be obtained on a second region of a test strip. Employing both of these tests substantially simultaneously on a urine sample disposed within a single container is a desirable and acceptable practice, as it has been found that neither test has any adverse effect on the validity and reliability of the other test. This approach, therefore, facilitates a physician in his office or a small clinic obtaining a single urine sample, dipping a test strip in the urine sample, waiting the appropriate amount of time for the result, and immediately being able to provide targeted medication. For example, if volume expansion hypertension is found to exist, a diuretic, such as a thiazide, may be given to a patient in an appropriate dosage well known to those skilled in the art. If vasoconstrictive hypertension has been found to exist, an angiotensin-converting enzyme (“ACE”) may be administered to the patient in a proper dosage which is well known to those skilled in the art. Alternate medications, which may be employed in treating vasoconstrictive hypertension, are angiotensinogen-receptive blockers (“ARB”) and direct renin antagonists.

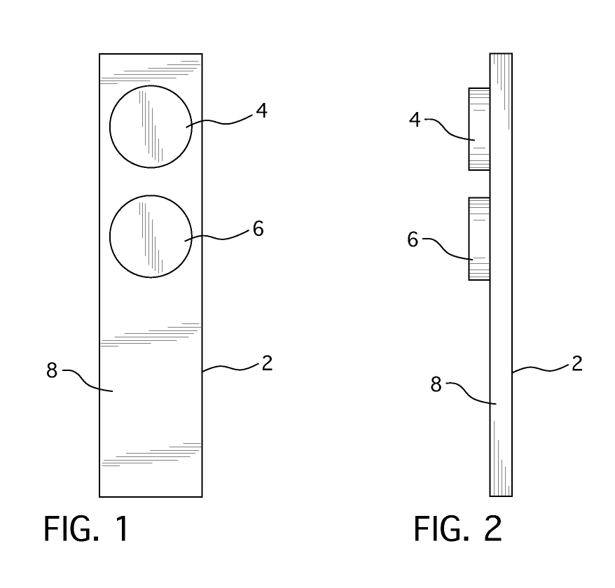

Referring to FIGS. 1 and 2, there is shown an elongated disposable test strip 2 which may be composed of any inert, structurally-sound material to which test regions may be secured by self adherence or by being separately fabricated and adhesively or otherwise bonded to the test strip 2. The test strip may, for example, have a length of about 4 inches to 6 inches, a width of about 3 inches to 4 inches, and a thickness of about ⅛ inch to ½ inch. It may be composed of a suitable synthetic, resinous material.

A first region 4 may be provided with the test materials to ascertain whether a particular level of bufodienolides is present, so as to confirm the existence of volume expansion hypertension in the patient. A second region 6 may have the test materials required to determine whether vasoconstrictive hypertension is present. If desired, the test regions 4, 6 may be recessed within strip 2. A free and gripping portion 8 is provided, so that one administering the test can grip the test strip at an adequate distance from the test regions 4, 6, so that the individual will not come in contact with the urine specimen. It is understood that conventional precautions, such as the wearing of latex gloves, would be employed. A suitable color comparison chart, such as is employed with a number of other tests and is well known to those skilled in the art, may be provided as a separate chart or, for example, on the label of the container in which the test strips are stored. Such a chart would have variations in color separately provided for each of the tests, so that one may, with the naked eye, whether or not enhanced by reading glasses, magnifying glasses, or other assists, may make a determination as to each category of hypertension.

In the alternative, the test regions 4, 6 may be evaluated using a suitably-programmed instrument, such as a colorimeter, for example, which would provide a precise determination of the level of the monitored constituents of the patient's urine specimen, and thereby provide a determination of as to whether either type of hypertension exists.

In employing the kit, a fresh urine specimen is obtained in a clean container. The strip is immersed in the urine specimen making sure that all of the test regions enter the test region. After a few seconds, the strip is removed, and after adequate time for a reaction to occur (typically, up to a few minutes), the strip is held against the color chart to compare the chart colors with the region colors in order to determine the test results. In the alternative, if an instrument is employed to do the reading, the instrument may display or provide a hard copy printout of the results or both.

While the example disclosed herein shows the use of a single region for each test, it will be appreciated that if additional compounds were to be monitored, additional regions could be added to the strip.

The test strip test regions involve a reaction between the substances (marinobufagenin and angiotensinogen) and antibodies specifically directed at each of those two compounds. This reaction may involve an ELISA-based assay.

The test strip regions may be of any desired configuration, and if circular, may have a diameter of about 1/16 inch to ¼ inch, and if the configuration is square, may be on the order of about 1/16 inch to ¼ inch per side.

It will be appreciated that the present invention provides an efficient, economical, and rapid means for substantially simultaneously determining if a patient has volume expansion hypertension or vasoconstrictive hypertension employing urine as the specimen. The present invention also permits employing the kit by a physician to prescribe immediately targeted medication for treatment of the patient.

### Example

Tests were performed to determine whether nonpregnant rats of one of two models of human essential hypertension would create a problem with respect to the other model. More specifically, a volume expansion model included taking out one kidney, thereby accelerating the process of developing hypertension, plus the replacement of drinking water with saline and weekly administration of DOCA, a powerful mineralocorticoid in order to ensure that the salt present in the saline is retained in the rat. This was compared with a model used to induce vasoconstriction by infusing a powerful vasoconstrictor, angiotensin II. It was theorized that the use of an antagonist of MBG, resibufogenin (“RBG”) would ameliorate the hypertension in volume expanded animals and would have no effect on the rats infused with angiotensin II. The results of these tests are shown in Table 1.

TABLE 1

| | | | No. of | |
|---|---|---|---|---|
| | U_{AGT} (Initial) | U_{AGT} (Final) | Samples | |
| Groups | (nmole/day) | (nmole/day) | (n) | P Values |
| Sham | 2.3 ± 0.2 | 2.4 ± 0.5 | 6 | |
| NDS | 2.1 ± 0.3 | 2.2 ± 0.3 | 5 | Sham vs NDS > 0.5 |
| NDSR | None | 2.0 ± 0.2 | 10 | Sham vs NDSR > 0.5 |
| ANG | 2.6 ± 0.2 | 7.1 ± 0.5 | 10 | Sham vs ANG < 0.001 |
| ANGR | 2.6 ± 0.2 | 7.8 ± 0.4 | 10 | Sham vs ANGR < 0.001 |

All Male Rats:
NDS = nephrectomy DOCA saline
NDSR = nephrectomy DOCA saline + RBG
ANG = angiotensin
ANGR = angiotensin + RBG

The test indicates the group of rats from a Sham (control) rat with measurements being taken initially and finally. It is noted that, in the NDS rats, which were subjected to nephrectomy and DOCA plus saline, there was no significant increase in the excretion of angiotensinogen. Similarly, in the NDSR rats, which were subjected to the nephrectomy and DOCA plus saline, plus being treated with resibufogenin to ameliorate the hypertension, there was no meaningful increase in angiotensinogen. By contrast, both the angiotensin II treated rats (ANG) and the angiotensin II treated rats who were treated with RBG (ANGR) experienced a very substantial increase in angiotensinogen excretion, thereby showing that the treatment with RBG had no effect on the angiotensinogen excretion as compared with the Sham rats. The RBG, as expected, also had no effect on the hypertension induced by angiotensin infusion.

In another embodiment of the present invention, a urine specimen is employed to determine the level of bufodienolide in the urine in order to determine if volume expansion hypertension exists in the patient. A preferred bufodienolide for use in this test is marinobufagenin. The test may be performed on either male patients or female patients.

Whereas particular embodiments of the present invention have been described herein for purpose of illustration, it will be evident to those skilled in the art that numerous variations of the details may be made without departing from the invention, as set forth in the appended claims.

What is claimed is:

1. A method of testing a patient for hypertension comprising
providing a urine specimen from said patient,
performing a first test on said urine specimen to determine if volume expansion hypertension exists by measuring the level of bufodienolides in said urine specimen,
performing a second test on said urine specimen to determine if vasoconstrictive hypertension exists by determining the level of angiotensinogen in said urine specimen, and
performing said first test and said second test on said urine specimen substantially simultaneously without each said test having an adverse effect on the accuracy of the other said test.

2. The method of testing a patient for hypertension of claim 1 including employing marinobufagenin as said bufodienolide.

3. The method of testing a patient for hypertension of claim 2 including
providing said urine specimen in a container, and
performing both said first test and said second test within said container.

4. The method of testing a patient for hypertension of claim 1 including
providing a test strip having a first region for effecting said first test and a second region for effecting said second test,
immersing said test strip in said urine specimen,
withdrawing said test strip from said urine specimen, and
determining whether volume expansion hypertension or vasoconstrictive hypertension exists by monitoring said first region and said second region.

5. The method of testing a patient for hypertension of claim 4 including effecting said monitoring visually.

6. The method of testing a patient for hypertension of claim 4 including effecting said monitoring by automated means.

7. The method of testing a patient for hypertension of claim 6 including
said automated means is a colorimeter.

8. The method of testing of claim 3 including
monitoring said first region and said second region for a color change which corresponds to volume expansion hypertension or vasoconstrictive hypertension.

9. A method of treating a patient for hypertension comprising
providing a urine specimen from said patient, performing a first test on said urine specimen to determine if volume expansion hypertension exists by measuring the level of bufodienolides in said urine specimen,
performing a second test on said urine specimen to determine if vasoconstrictive hypertension exists by determining the level of angiotensinogen in said urine specimen,
performing said first test and said second test on said urine specimen substantially simultaneously without each said test having an adverse effect on the accuracy of the other said test, and
if either volume expansion hypertension or vasoconstrictive hypertension is determined to exist, treating said patient for said condition.

10. The method of treating a patient for hypertension of claim 9 including
employing marinobufagenin as said bufodienolide.

11. The method of treating a patient for hypertension of claim 9 including
if volume expansion hypertension is found to exist, treating said patient with a diuretic.

12. The method of treating a patient for hypertension of claim 9 including
if vasoconstrictive hypertension has been found to exist, treating said patient by a pharmaceutical agent selected from the group consisting of an ACE inhibitor, angiotensin receptor blocker, and a direct renin inhibitor.

13. The method of treating a patient for hypertension of claim 9 including
providing said urine specimen in a container, and
performing both said first test and said second test within said container.

14. The method of treating a patient for hypertension of claim 9 including
providing a test strip having a first region for effecting said first test and a second region for effecting said second test,
immersing said test strip in said urine specimen,
withdrawing said test strip from said urine specimen, and
determining whether volume expansion hypertension or vasoconstrictive hypertension exists by monitoring said first region and said second region.

15. The method of treating a patient for hypertension of claim 9 including
effecting said monitoring visually.

16. The method of treating a patient for hypertension of claim 9 including
effecting said monitoring by automated means.

17. The method of treating a patient for hypertension of claim 9 including
monitoring said first region and said second region for a color change which corresponds to volume expansion hypertension or vasoconstrictive hypertension.

18. A test kit for testing a patient for hypertension comprising
an elongated test strip having at least one first region for performing a first test on a urine specimen to determine if volume expansion hypertension exists in the patient by measuring the level of marinobufagenin in said urine specimen, and
at least one second region for performing a second test on said urine specimen to determine if vasoconstrictive hypertension exists by measuring the level of angiotensinogen in said urine specimen.

19. The test kit for testing a patient for hypertension of claim 18 including
a color chart for comparison with said first region and said second region in determining, respectively, whether volume expansion hypertension or vasoconstrictive hypertension exists.

20. The test kit for testing a patient for hypertension of claim 19 including
said first region structured to effect a predetermined change in color in the event that said first test results in a determination of the assistance of volume expansion hypertension, and
said second region structured to effect a predetermined change in color in the event that said second test determines the existence of vasoconstrictive hypertension.

21. A method of testing a patient for volume expansion hypertension comprising
providing a urine specimen from said patient,
measuring the level of bufodienolide in said urine specimen, and
determining if volume expansion hypertension exists on the basis of the level of bufodienolide in said urine specimen.

22. The method of testing a patient for volume expansion hypertension of claim 21 including marinobufagenin as said bufodienolide.

23. The method of testing a patient for volume expansion hypertension of claim 22 including employing said test on a male patient.

24. The method of testing a patient for volume expansion hypertension of claim 22 including employing said test on a female patient.
